(19) United States
(12) Patent Application Publication
(10) Pub. No.: US 2017/0049726 A1
(43) Pub. Date: Feb. 23, 2017

(54) METFORMIN IN TREATMENT OF NEUROBLASTOMA

(71) Applicant: UNIVERSITY OF SOUTH CAROLINA, Columbia, SC (US)

(72) Inventors: Ambrish Kumar, Columbia, SC (US); Ugra Sen Singh, Columbia, SC (US); Donald J. Dipette, Blythewood, SC (US)

(21) Appl. No.: 15/243,204

(22) Filed: Aug. 22, 2016

Related U.S. Application Data
(60) Provisional application No. 62/208,145, filed on Aug. 21, 2015.

(51) Int. Cl. A61K 31/155; A61K 45/06

(52) U.S. Cl. CPC A61K 31/155; A61K 45/06

(57) ABSTRACT

Use of metformin in inhibition of growth and/or viability of human neuroblastoma cells is described. Use of metformin in inhibition of growth and/or viability of neuroblastoma cancer stem cells is also described. The metformin is shown to be effective both in vitro and in vivo in inhibition of growth and/or viability of neuroblastoma cells of multiple different genetic backgrounds.

### CROSS REFERENCE TO RELATED APPLICATION

This application claims filing benefit of U.S. Provisional Patent Application Ser. No. 62/208,145 having a filing date of Aug. 21, 2015, which is incorporated herein in its entirety.

### BACKGROUND

The majority of cancer therapies available are generally developed for adults and their efficacy in treating childhood cancers is at best unknown. From 1948 to 2003, regulators approved 120 new cancer therapies, but only 15 contained information about pediatric use. Moreover, pediatric cancers differ significantly from adult cancers in terms of their site of origin and cause of occurrence. Standard chemotherapy and radiation therapy are used to treat pediatric cancer but unfortunately, these methods can have long-term side effects. As such, children who survive cancer will need careful attention for the rest of their lives. There is urgent need to develop safer drugs and therapeutic approaches specific for the treatment of childhood cancers.

Of common childhood cancers, neuroblastoma very often presents in childhood and is the most common cancer in babies younger than one and the second most common tumor in children. In the United States, approximately 700 children are diagnosed with neuroblastoma each year. It accounts for 7% of all childhood cancers, and is responsible for 15% of all cancer deaths in children younger than 15 years.

Neuroblastoma is a malignant cancer of the postganglionic sympathetic nervous system that derives from the neural crest cells during embryonic development. Initially it develops in the adrenal gland and later spreads to various body organs including the liver, bone, bone marrow, lymph nodes, neck and chest. Even with therapy including chemotherapy, surgery, and radiation, the mortality rate remains high as various genetic and cytogenetic alterations can allow the neuroblastoma tumors to develop drug resistance. The five-year survival rate for children with low-risk neuroblastoma is higher than 95%, and for children with intermediate-risk neuroblastoma the survival rate is 80% to 90%, but long-term survival drops to only about 30% to 50% of children with high-risk neuroblastoma.

Neuroblastoma contains a heterogeneous population of cells that originates from embryonic neural crest cells. The majority of tumor cells present in neuroblastoma are N-type cells (neuroblastic), S-type cells (substrate adherent) and I-type cells (intermediate), each of which differ morphologically and biochemically. I-type cells are intermediate cells as these cells can differentiate into N-type as well as S-type neuroblastoma cells. I-type cells are considered as neuroblastoma stem cells since they are multipotent and differentiate to both N- or S-type of cells and because they express stem cell markers CD133 and c-kit (CD117).

Stem cells possess two primary properties: i—Self-renewal (the ability to go through numerous cycles of cell division while maintaining the undifferentiated state), and ii—Potency (the capacity to differentiate into specialized cell types). By definition cancer stem cells (CSCs) are cells within a tumor that have the ability for self-renewal and regeneration of heterogeneous lineages of cancer cells that comprise the tumor. Multiple mutations in oncogenes, tumor suppressor genes or epigenetic modifications transform normal stem cells into CSCs. As a result these cells acquire oncogene induced plasticity. The presence of CSCs has been reported in various cancers such as breast cancer, melanoma, leukemia, glioblastoma, colon cancer, pancreatic cancers etc. CSCs are significantly different from normal stem cells as CSCs can form tumors when transplanted into animals, unlike normal stem cells. It has been reported that cancer cells having stem cell-like characteristics develop drug resistance; hence CSC-based therapy for the treatment of various types of cancer has gained a lot of attention in recent years. According to recent theory, tumors are comprised of normal cancer cells with small population of cancer stem cells. The currently available therapies kill normal cancer cells. However, CSCs escapes these therapies and further regrow to tumors. The therapies aiming to CSCs could completely eliminate the tumor initiating cells and eradicate the cancer (FIG. 1).

The existence of drug resistance in combination with the inability of conventional therapy to target CSCs makes cancer treatment, and more specifically neuroblastoma treatment extremely challenging, particularly when treating childhood cancers.

What is needed in the art are targeted agents and combination chemotherapy in the treatment of neuroblastoma.

### SUMMARY

According to one embodiment, a method for inhibiting the growth and/or viability of neuroblastoma cells is described. In one particular embodiment, a method can be directed to neuroblastoma stem cells. For example, the method can include contacting neuroblastoma cells with metformin. In one embodiment, disclosed is a method for treatment of neuroblastoma that includes administering metformin to a person suffering from neuroblastoma, and in one particular embodiment to a child suffering from neuroblastoma.

### BRIEF DESCRIPTION OF THE FIGURES

The present disclosure may be better understood with reference to the figures in which:

FIG. 1 schematically illustrates a comparison between stem cell-based therapy and conventional therapy for cancer treatment.

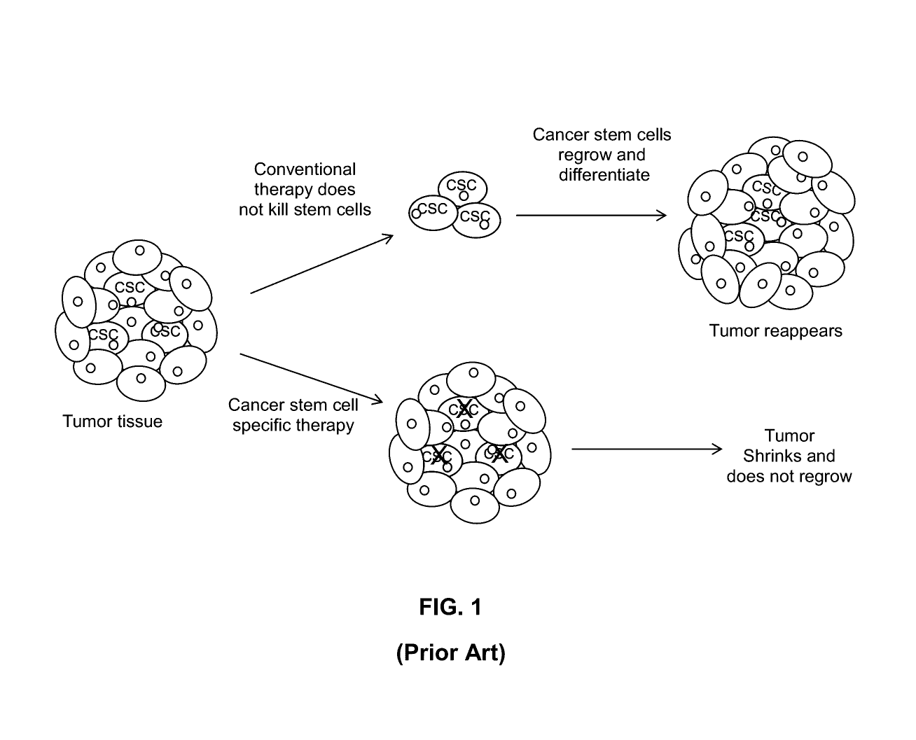

FIG. 2 presents representative Western blots showing expression of stem cell marker proteins in neuroblastoma cells. Equal amounts of total cell proteins (100 μg) from neuroblastoma cell lines of different genetics (SH-SY5Y, SK-N-BE(2), IMR-32, NGP and SK-N-F1) were separated on SDS-polyacrylamide gel, transferred on PVDF membrane, and probed with antibodies raised against stem cell specific transcription factors-sox2, oct4 and nanog. Membranes were re-probed with β-actin to check loading difference.

FIG. 3 presents immunofluorescence images showing expression of sox2, oct4 and nanog in SK-N-BE(2) neuroblastoma cells. The cells were fixed with 4% paraformaldehyde/PBS, permeabilized and incubated with primary antibodies for sox2, oct4 and nanog proteins. Phalloidin and DAPI were used to stain cell cytoskeleton and cell nucleus, respectively. The expression of sox2, oct4 and nanog proteins were detected in the nucleus, and overlapped with DAPI stain (single and merged images). Normal IgG was used as a negative control to evaluate the specific staining of sox2, oct4 and nanog antibodies. Scale bar=100 μm.

FIG. 4 presents western blots illustrating the effect of metformin on sox2, oct4 and nanog protein expression from SK-N-BE(2) cells. Total proteins (100 μg) from SK-N-BE(2) cells treated for 2 days with different concentrations of metformin (1, 10 and 20 mM) were separated on SDS-polyacrylamide gel and probed with sox2, oct4 and nanog antibodies. Blots demonstrated that metformin reduced the expression of sox2 and nanog proteins, not of oct4 protein, in a dose-dependent manner.

FIG. 5 demonstrates that metformin induced apoptotic cell death in neuroblastoma stem cells. SK-N-BE(2) cells grown as a monolayer in LabTekII chamber slides were treated with metformin (10 mM) for 2 days, fixed with 4% paraformaldehyde/PBS, permeabilized and labeled with sox2 and cleaved caspase-3 antibodies. DAPI staining was used to detect nucleus. The signals of sox2 and cleaved caspasre-3 were observed in cell nucleus and cytoplasma, respectively (see single images). The presence of cleaved caspase-3 signals (cytoplasmic) in sox2-expressing cells (nuclear) cells confirmed that metformin induced apoptotic cell death in cancer stem cells (merged images).

FIG. 6 presents bright contrast images showing the morphology of neuroblastoma cells grown in different cell culture mediums. In complete cell culture medium (DMEM+10% fetal bovine serum), neuroblastoma cells were grown as monolayer (upper panels). When neuroblastoma cells were transferred in serum-free stem cell specific medium (DMEM/F12 supplemented with bFGF, EGF, N-2 and B27 supplement), tumor cells start de-differentiating and start growing as spheroids enriched in cancer stem cells (middle panels). Spheroids were again differentiated in complete cell culture medium (DMEM with 10% fetal bovine serum) and proliferated as monolayer (lower panels). These data indicated that neuroblastoma cells possess stem cell like features.

FIG. 7 illustrates that metformin reduced the self-renewal and differentiation potential of SK-N-BE(2) stem cells. Bright contrast images (A) show primary spheroid formation in the absence (control) or presence of metformin (0.1, 0.5, 1, 10 and 20 mM). Pictures were taken on day 20, and number of spheroids 100 μm in size) in fold change was plotted (B) (*p<0.05 vs control). The primary spheroids were dissociated by trypsinization and further grown in ultra-low attachment 6-well plates in serum-free stem cell specific medium (with or without metformin). Formed secondary spheroids were counted and plotted (*p<0.05 vs control) (C).

FIG. 8 presents images demonstrating that metform in promotes apoptotic cell death in neuroblastoma spheroids. Representative immunofluorescence images showing the signals of cleaved caspase-3 (a marker of apoptosis) in sox2-expressing cells in metform in-treated SK-N-BE(2) spheroids. Stem cell transcription factor sox2 was used to detect stem cells in spheroids. DAPI stained nucleus and overlapped with sox2 signals. Scale bar=100 μm.

FIG. 9 illustrates that metform in inhibits the growth of tumors generated from SK-N-BE(2) spheroids in mice. 1×10⁵ tumor cells from SK-N-BE(2) spheroids (15-day-old) were injected subcutaneously in the flank region of athymic nude mice (nu/nu). When the palpable tumor was visible, metformin (100 mg/kg body weight) was given daily by oral gavage. Tumor volume was measured after every 4 days. At the end of experiments, mice were sacrificed, tumors were collected and photographed as illustrated.

FIG. 10 demonstrates that metform in induces activation of caspase-3 in tumors generated from SK-N-BE(2) spheroids. Representative immunofluorescence images show sox2 and cleaved caspase-3 staining in SK-N-BE(2) tumors. Nuclei were counterstained with DAPI. The cytoplasmic cleaved caspase-3 staining (single image) was observed in tumors treated with metform in (lower panel). The signals for sox2 were present in nucleus and overlapped with DAPI stain. In metform in-treated tumors, cleaved caspase-3 signals were present in sox2-expressing cells indicating that metformin induced cell death in stem cells also (enlarge merged images). In tumors collected from metformin-untreated control mice, no such cleaved caspase-3 staining was observed (upper panel). Scale=100 μm.

### DETAILED DESCRIPTION

The following description and other modifications and variations to the present invention may be practiced by those of ordinary skill in the art, without departing from the spirit and scope of the present invention. In addition, it should be understood that aspects of the various embodiments may be interchanged both in whole and in part. Furthermore, those of ordinary skill in the art will appreciate that the following description is by way of example only, and is not intended to limit the invention.

According to the present disclosure, metformin has been found efficacious in treatment and study of neuroblastoma. In one embodiment, the present disclosure is directed to the utilization of metformin in inhibiting the survival of human neuroblastoma cells of different genetic background such as SH-SY5Y, SK-N-BE(2), IMR-32, NGP and SK-N-F1 neuroblastoma cells. In one particular embodiment, a method can be directed to neuroblastoma stem cells.

Despite focusing on new molecular targets and the use of multimodal therapy which includes surgery, radiotherapy in conjunction with chemotherapy and monoclonal antibody based immunotherapy; approximately 30% of children with high-risk neuroblastoma remain incurable. Hence, the utilization of metformin as a therapeutic compound in treatment of neuroblastoma is needed, particularly as this compound can exhibit less toxicity as compared to many previously known chemotherapeutic agents.

As described in more detail further herein, metformin can dose-dependently reduce the protein level of multiple neuroblastoma stem cell-specific transcription factor proteins. Without wishing to be bound by any particular theory, it is believe that this leads to inhibition of the formation of neuroblastoma spheroids; the formation of which is typical for neuroblastoma cells in the presence of serum-free stem cell specific medium.

Moreover, neuroblastoma tumors generated from stem cells can be reduced in size when subjects are treated with metformin. Specifically, metformin can induce apoptosis by activating caspase-3. In fact, the presence of cleaved caspase-3 signal in both sox2-expressing and sox2-nonexpressing cancer cells (as determined by immunohistochemistry) indicates that metformin can induce cell death not only in normal neuroblastoma tumor cells but also in neuroblastoma tumor stem cells.

Metformin (Chemical name: N′,N′-dimethylbiguanide) is a dimethyl biguanide having the general structure:

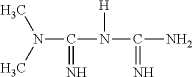

According to the present disclosure, metform in or a derivative thereof can be utilized in treatment of neuroblastoma. Metformin and derivatives thereof have been disclosed previously, for instance in treatment of type II diabetes mellitus as well as certain other disease conditions. As utilized herein, the term “metformin” can generally refer to the above structure as well as any biguanide species thereof as is generally known in the art. For instance, in one embodiment a method can encompass a pharmaceutically acceptable salts of the general structure:

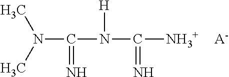

in which A is the anion of the non-toxic salt. Pharmaceutically acceptable salts of metformin can include, without limitation, phosphate, sulfate, hydrochloride, salicylate, maleate, benzoate, ethanedisulfonate, fumarate and glycolate salts. In one particular embodiment, the metform in can be 1,1-dimethylbiguanide hydrochloride (metform in HCl). Examples of suitable forms of metform in encompassed herein can include those described in U.S. Pat. No. 3,174,921 to Sterne; U.S. Pat. No. 6,031,004 to Timmins; U.S. Pat. No. 6,890,957 to Chandran, et al.; and U.S. Pat. No. 9,416,098 to Kim, et al., all of which being incorporated herein by reference. For instance, metformin can encompass the crystal form, hydrates, solvats, diastereomers or enantiomers.

Metformin can be given in dosage generally varying from about 250 mg to 3000 mg, particularly from 500 mg to 2000 mg up to 2500 mg per day using various dosage regimens. The unit dosage strengths of the metformin hydrochloride for use in the present invention may be from 100 mg to 2000 mg or from 250 mg to 2000 mg, preferably from 250 mg to 1000 mg. Particular dosage strengths may be 250, 500, 625, 750, 850 or 1000 mg of metformin hydrochloride. More particular unit dosage strengths of metformin hydrochloride for incorporation into the fixed dose combination pharmaceutical compositions of the present invention are 500, 850 and 1000 mg of metformin hydrochloride. Of course, dosage can vary as is known in the art, for instance based upon subject body weight, the specific state of the neuroblastoma, etc. For instance, a dosage amount for a patient can be from about 5 mg/kg/day to about 20 mg/kg/day, for instance about 10 mg/kg/day in one embodiment.

It should be understood that the metformin can be combined with other materials as are known in the art in forming a treatment or study composition. Metformin is a starting compound that can be used either alone or in combination with other compounds for use as described herein. A treatment or study composition is not intended to be limited to containing only metformin.

Without wishing to be bound to any particular theory, it is believed that the reduced viability by metformin affects the neuroblastoma cells' ability to form compact spheroids, which decreases the tumorigenic potential of both N-myc amplified (e.g., SK-N-BE(2) cells) and N-myc non-amplified neuroblastoma cells (e.g., SH-SY5Y cells).

The inhibitory effect of metformin on cell growth and viability is believed to result from caspase-mediated cell death. As is known, increased level of the activated form of caspase-3 has been shown to trigger DNA fragmentation, chromatin condensation, membrane blebbing and cell shrinkage that leads to the programme cell death (apoptosis). Metformin-treated neuroblastoma cells can exhibit higher levels of apoptosis as compared to control cells, and indications are that metformin triggers apoptotic cell death pathways via activation of a caspase cascade.

The methods can be utilized in vivo for treatment of neuroblastoma or in vitro for study of neuroblastoma cells. According to an in vivo treatment method, a composition including methformin (e.g., metformin HCl) and a pharmaceutically compatible carrier can be delivered to a patient via any pharmaceutically acceptable delivery system. For instance, a composition including a pharmaceutically compatible carrier and metformin may be a solid, liquid or aerosol form and may be administered by any known pharmaceutically acceptable route of administration.

A non-limiting exemplary listing of possible solid compositions can include pills, creams, and implantable dosage units. An implantable dosage unit can, in one embodiment, be administered locally, for example at a tumor site, or can be implanted for systemic release of the composition, for example subcutaneously. A non-limiting exemplary listing of possible liquid compositions can include formulations adapted for injection subcutaneously, intravenously, intra-arterially, and formulations for topical and intraocular administration. Possible examples of aerosol formulations include inhaler formulations for direct administration to the lungs.

Compositions can generally be administered by standard routes. For example, the compositions may be administered by topical, transdermal, intra-peritoneal, intracranial, intra-cerebroventricular, intra-cerebral, intra-vaginal, intrauterine, oral, rectal or parenteral (e.g., intravenous, intra-spinal, subcutaneous or intramuscular) route. Osmotic mini-pumps may also be used to provide controlled delivery of metformin through cannulae to the site of interest, such as directly into a metastatic growth.

Pharmaceutical compositions for parenteral injection can include pharmaceutically acceptable sterile aqueous or nonaqueous solutions, dispersions, suspensions or emulsions as well as sterile powders for reconstitution into sterile injectable solutions or dispersions just prior to use. Examples of suitable aqueous and nonaqueous carriers, diluents, solvents or vehicles include water, ethanol, polyols (e.g., glycerol, propylene glycol, polyethylene glycol and the like), carboxymethylcellulose and suitable mixtures thereof, vegetable oils (e.g., olive oil) and injectable organic esters such as ethyl oleate. A composition can contain minor amounts of auxiliary substances such as wetting or emulsifying agents, pH buffering agents and the like that can enhance the effectiveness of the active ingredient. Proper fluidity may be maintained, for example, by the use of coating materials such as lecithin, by the maintenance of the required particle size in the case of dispersions and by the use of surfactants. A composition may also contain adjuvants such as preservatives, wetting agents, emulsifying agents and dispersing agents. Prevention of the action of microorganisms may be ensured by the inclusion of various antibacterial and antifungal agents such as paraben, chlorobutanol, phenol, sorbic acid and the like. It may also be desirable to include isotonic agents such as sugars, sodium chloride and the like.

Prolonged absorption of an injectable pharmaceutical form may be brought about by the inclusion of agents, such as aluminum monostearate and gelatin, which can delay absorption. For example, injectable depot forms can be made by forming microencapsule matrices of the metformin in biodegradable polymers such as polylactide-polyglycolide, poly(orthoesters) and poly(anhydrides). Depending upon the ratio of metformin to polymer and the nature of the particular polymer employed, the rate of release can be controlled. Depot injectable formulations can also be prepared by entrapping the metformin in liposomes or microemulsions that are compatible with body tissues. An injectable formulation may be sterilized, for example, by filtration through a bacterial-retaining filter or by incorporating sterilizing agents in the form of sterile solid compositions that can be dissolved or dispersed in sterile water or other sterile injectable media just prior to use.

A composition can include pharmaceutically acceptable salts of the components therein, e.g., those that may be derived from inorganic or organic acids. Pharmaceutically acceptable salts are well known in the art. Pharmaceutically acceptable salts include the acid addition salts (formed with the free amino groups of the polypeptide) that are formed with inorganic acids such as, for example, hydrochloric or phosphoric acids, or such organic acids as acetic, tartaric, mandelic and the like. Salts formed with the free carboxyl groups can also be derived from inorganic bases such as, for example, sodium, potassium, ammonium, calcium or ferric hydroxides, and such organic bases as isopropylamine, trimethylamine, 2-ethylamino ethanol, histidine, procaine and the like. Representative acid addition salts include, but are not limited to acetate, adipate, alginate, citrate, aspartate, benzoate, benzenesulfonate, bisulfate, butyrate, camphorate, camphorsulfonate, digluconate, glycerophosphate, hemisulfate, heptonoate, hexanoate, fumarate, hydrochloride, hydrobromide, hydroiodide, 2-hydroxymethanesulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl, and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which may be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

A treatment method can include use of timed release or sustained release delivery systems as are generally known in the art. A sustained-release matrix can include a matrix made of materials, usually polymers, which are degradable by enzymatic or acid/base hydrolysis or by dissolution. Once inserted into the body, such a matrix can be acted upon by enzymes and body fluids. The sustained-release matrix desirably is chosen from biocompatible materials such as and without limitation to liposomes, polylactides (polylactic acid), polyglycolide (polymer of glycolic acid), polylactide co-glycolide (co-polymers of lactic acid and glycolic acid) polyanhydrides, poly(ortho)esters, polyproteins, hyaluronic acid, collagen, chondroitin sulfate, carboxylic acids, fatty acids, phospholipids, polysaccharides, nucleic acids, polyamino acids, amino acids such as phenylalanine, tyrosine, isoleucine, polynucleotides, polyvinyl propylene, polyvinylpyrrolidone and silicone.

When metformin is administered orally, the therapeutic compositions can be in the form of a tablet, capsule, powder, solution or elixir. When administered in tablet form, a composition may additionally contain a solid carrier such as a gelatin or an adjuvant. A tablet, capsule, or powder can, for example, contain from about 5 to 95% by weight of metformin. In one embodiment, a composition can contain from about 25 to 90% by weight of metformin.

When administered orally in liquid form, a liquid carrier such as water, petroleum, oils of animal or plant origin such as peanut oil, mineral oil, soybean oil, or sesame oil, or synthetic oils may be added. A liquid form may further contain physiological saline solution, dextrose or other saccharide solution, or glycols such as ethylene glycol, propylene glycol or polyethylene glycol. When administered in liquid form, a composition can contain from about 0.5 to 90% by weight metformin, in one embodiment from about 1 to 50% by weight metformin.

When an effective amount of metformin is administered by intravenous, cutaneous or subcutaneous injection, the metformin composition can generally be in the form of a pyrogen-free, parenterally acceptable aqueous solution. The preparation of such parenterally acceptable solutions, having due regard to pH, isotonicity, stability, and the like, is within the skill in the art. A preferred pharmaceutical composition for intravenous, cutaneous, or subcutaneous injection can contain, in addition to the metformin, an isotonic vehicle such as Sodium Chloride Injection, Ringer's Injection, Dextrose Injection, Dextrose and Sodium Chloride Injection, Lactated Ringer's Injection, or other vehicle as known in the art. The composition may also contain stabilizers, preservatives, buffers, antioxidants, or other additives known to those of skill in the art.

It is to be understood that the in vivo methods have application for both human and veterinary use. The methods of the present invention contemplate single as well as multiple administrations, given either simultaneously or over an extended period of time. In addition, metformin can be administered in treatment of neuroblastoma in conjunction with other forms of therapy, e.g., and without limitation, chemotherapy, radiotherapy, or other immunotherapy, surgical intervention, and combinations thereof.

Removal of neuroblastoma stem cells in tumors by use of the disclosed methods can prevent the further occurrence and progression of cancer cells. Moreover metformin can be effectively utilized to inhibit growth and development of neuroblastoma without affecting the survival of undifferentiated human normal stem cells (e.g. umbilical cord-derived mesenchymal stem cells), primary cortical neurons, and various normal endothelial cells such as human microvascular endothelial cells (HMEC-1) and primary endothelial cells from either human umbilical vein or bovine aorta as metformin can be highly selective to neuroblastoma cells. The success of any therapeutic compound to use as a drug in disease treatments depends on the facts that it should be safe, cost-effective, and should be available easily. The facts that metformin is cost effective, safe and already approved by United States Food and Drug Administration (FDA) to treat type 2 diabetes in children further strengthen metformin as a therapeutic agent can be used to treat pediatric cancers, especially neuroblastoma.

The present disclosure may be better understood with reference to the Example, set forth below.

### Example

### Human Neuroblastoma Cell Lines and Spheroid Culture

SH-SY5Y, IMR-32, SK-N-BE(2) and SKNF-1 human neuroblastoma cells were purchased from The American Type Culture Collection (ATCC; Manassas, Va.), and NGP cells were kind gift from Garrett M. Brodeur (The Children's Hospital of Philadelphia, Philadelphia, Pa.). Cells were maintained as monolayer in Dulbecco's Modified Eagle's Medium (DMEM) containing 10% fetal bovine serum (FBS; Atlanta Biologicals, Lawrenceville, Ga.) and 1% antibiotic-antimycotic solution (100 unit/ml penicillin, 100 mg/ml streptomycin and 0.25 mg/ml amphotericin B; Gibco, Grand Island, N.Y.) at 37° C. in a humidified incubator with 5% CO₂. For all the experiments, cell culture passages from 4-12 were used.

For spheroid formation, adherent cells were trypsin digested (0.25% Trypsin-EDTA) for 3-4 min at 37° C., washed and suspended in the serum-free stem cell specific medium [DMEM/F-12 medium (Lonza, Switzerland) supplemented with 1% N-2 supplement, 2% B-27 without vitamin A, 20 ng/ml human recombinant epidermal growth factor (hrEGF), 20 ng/ml human recombinant basic fibroblast growth factor (bFGF) and 1% antibiotic-antimycotic solution; all from Gibco]. The dispersed single cells were plated at density of 1,000 cells/well in Costar ultra-low attachment six-well plates (Corning Inc., Corning, N.Y.), and treated with indicated concentrations of metformin. The stock solution of metformin (MP Biomedicals, Solon, Ohio) was freshly prepared in sterile triple distilled water before each experiment. Cells treated with equal volume of vehicle were used as control. Cell culture medium was changed on every fourth day. For secondary spheroids formation, primary spheroids (20-day-old and ≧100 μm in size) were dissociated with 0.25% trypsin-EDTA and single cell suspensions were re-seeded at a density of 1,000 cells/well in the serum-free stem cell specific medium (presence or absence of metformin) in ultra-low attachment six-well plates, and allowed them to form secondary spheroids.

### Total Cell Protein Isolation and Western Blot Analyses

Cells, either from monolayer or from spheroids, were dissociated by trypsinization, washed with 1× phosphate buffered saline (PBS), pH 7.5, and lysed with 1× cell lysis buffer (Cell Signaling Technology, Danvers, Mass.) containing phenylmethylsulfonyl fluoride (PMSF) and protease inhibitors (aprotinin and leupeptin) for 30 min on ice. Cell lysate was centrifuged at 12000×rpm for 10 min at 4° C., and supernatant (total cell protein) was stored at −80° C. Protein concentration was determined by bicinchoninic acid method using BCA protein assay kit (Pierce/Thermo-scientific, Waltham, Mass.). Equal amount of proteins were diluted with 5×Laemmli samples loading buffer, boiled for five minutes, separated on sodium dodecyl sulfate (SDS)-polyacrylamide gel, and analyzed by Western blotting. Briefly, after electrophoresis, proteins were transferred on polyvinylidene difluoride (PVDF) membrane at 100 volt for 3 h in cold room. Membrane was blocked with 5% non-fat dry milk/TBST (20 mM Tris-CI, pH 7.4; 150 mM NaCl with 0.1% Tween-20) for 4 h at room temperature followed by incubation in primary antibodies diluted in 2.5% non-fat dry milk/TBST for overnight at 4° C. After washing with TBST, membrane was incubated with secondary antibodies (horseradish peroxidase-conjugated goat anti-rabbit or goat anti-mouse IgG; BioRad, Hercules, Calif.) diluted in 2.5% non-fat dry milk/TBST for 3 h at room temperature. Signals were detected by chemiluminiscence detection kit (Pierce/Thermo Scientific). Primary antibodies used were sox2, nanog, oct4, cleaved caspase-3 and β-actin (all from Cell Signaling Technology).

### In Vivo Xenograft Experiments

SK-N-BE(2) neuroblastoma spheroids (15-day-old) were dissociated by trypsinization, washed with stem cell specific medium and counted. 1×10⁵ cells were mixed (1:1 volume) with EGF-reduced matrigel (BD Bioscience, San Jose, Calif.) to make total volume of 0.1 ml. The cell mixture (0.1 ml) was injected subcutaneously (s.c.) into flanks of 6-week-old female nude athymic mice (nu/nu mice; Charles River Laboratories, Wilmington, Mass.). The animals were housed at 25° C. with a 12-h light/12-h dark cycle in laminar flow cabinets under specific pathogen-free conditions and given sterile water and food ad libitum. Engrafted mice were inspected for tumor appearance, and tumor growth was measured on every fourth day using a caliper. Tumor volume was calculated using the ellipsoid formula (length×width×height×0.5). All animal work was carried out according to experimental protocols approved by the Institutional Animal Care and Use Committee (IACUC) of the University of South Carolina.

When palpable tumor was visible (˜10 days after inoculation), mice were divided into two groups—(i) control without metformin, n=4; (ii) with metformin at dose 100 mg/kg body weight/mice, n=4. Metformin, dissolved in 200 μl sterile water, was given daily by oral gavage. When the tumor reached terminal size (≧1000 mm³) in metformin-untreated control group, mice from all groups were euthanized, tumors were harvested and weighed. Portions of tumor were fixed in 4% paraformaldehyde/PBS or snap frozen in liquid nitrogen for further use in biochemical assays.

### Immunofluorescence Imaging

Neuroblastoma cells grown in Lab-TekII chamber slides (Fisher Scientific, Pittsburgh, Pa.) were treated with metformin in complete culture medium. After treatments, cells were washed with 1×PBS, fixed with 4% paraformaldehyde/PBS for 20 min, and incubated in 1×PBS buffer containing 0.1M glycine and 0.3% Triton X-100 for 30 min at room temperature. Cells were washed with 1×PBS for 4 times (10 min each) and blocked with 5% immunoglobulin (IgG)-free bovine serum albumin (BSA; Jackson ImmunoResearch Laboratories, West Grove, Pa.) in 1×PBS for overnight at 4° C. Cells were incubated with antibodies raised against sox2, oct4 and nanog, and active form of caspase-3 diluted in 2.5% BSA+0.3% Triton X-100 in PBS for overnight at 4° C. Primary antibodies were detected with secondary antibodies conjugated with AlexaFluor-488 or AlexaFluor-568 for 2 h at room temperature. After washing with 1×PBS, cells were mounted with antifade Vectashield™ mounting media (Vector Laboratories, Burlingame, Calif.), and signals were visualized under Nikon™-E600 fluorescence microscope. Nuclei were stained with 4′, 6-diamidino-2-phenylindole (DAPI; Sigma).

For tumor tissue, paraformaldehyde-fixed paraffin-embedded tumor sections (5 μm thick) were deparaffinized in xylene, rehydrated with sequential immersion in graded ethanol (100%, 95%, 85%, 70% and 50%). Antigen unmasking was carried out by boiling slides in 10 mM sodium citrate buffer, pH 6.0, at 95° C. for 30 min. After cooling down at room temperature, sections were incubated with 0.1M glycine+0.3% Triton X-100/PBS for 30 min, and were blocked with 10% IgG-free BSA in 1×PBS for overnight at 4° C. Sections were incubated with primary antibodies diluted in 5% IgG-free BSA+0.3% Triton X-100/PBS (1:100 dilution) for overnight at 4° C. Primary antibodies were detected as described in previous paragraph. Primary antibodies used were: sox2, oct4 and nanog, and active form of caspase-3.

### Data Analysis

Data are presented as the mean and standard deviation (S.D.) of at least three independent experiments. Comparisons were made among the groups using one-way ANOVA followed by Tukey-Kramer ad hoc test (GraphPad software, La Jolla, Calif.). A p value<0.05 was considered significant.

### Neuroblastoma Cells Lines Express Stem Cell Markers

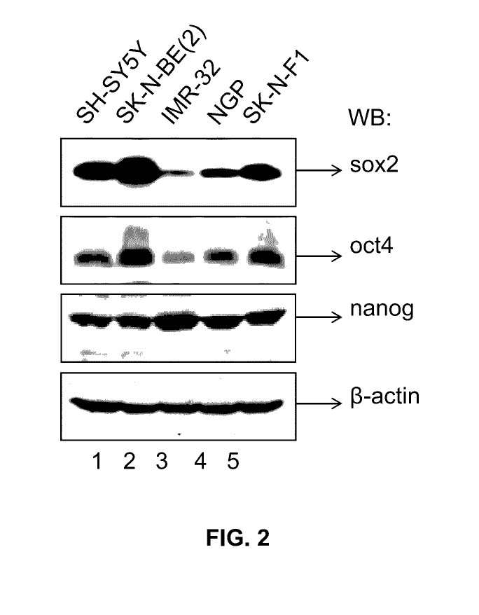

To test whether neuroblastoma cell lines include cancer stem cells, Western blotting and immunofluorescence staining were performed to detect the levels of stem cell marker proteins. In general, proteins sox2, oct4 and nanog transcription factors are the markers used to determine the presence of stem cells. A panel of neuroblastoma cells varying genetically was used including SH-SY5Y and SK-N-F1 that are MYCN non-amplified neuroblastoma cell lines and SK-N-BE2, NGP, and IMR-32 that are MYCN amplified neuroblastoma cell lines. Total cell protein was prepared from these cell lines, and expression of stem cell specific proteins—sox2, oct4, and nanog was detected by Western blot analysis. Blots (FIG. 2) showed that the expression of sox2, oct4 and nanog proteins was present in all neuroblastoma cell lines. This indicated that stem cells are present in these cell lines irrespective of their genetic variations.

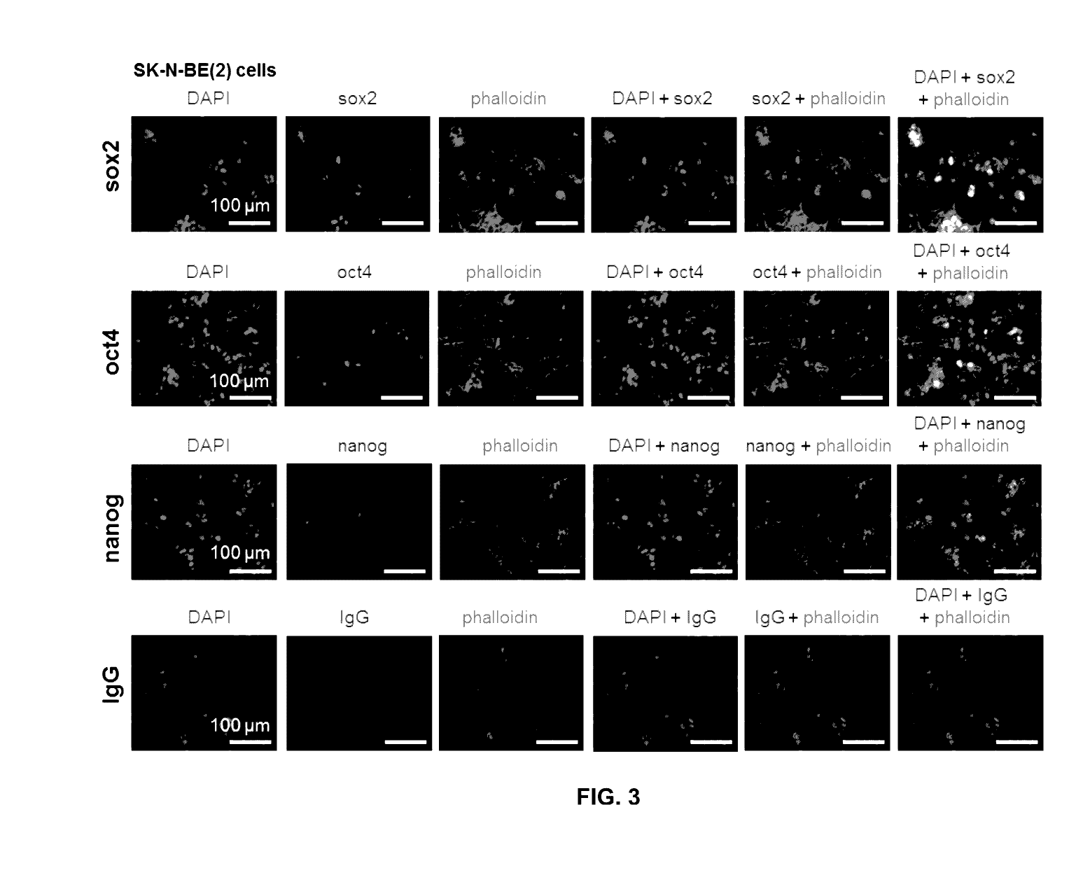

The expression of the above stem cell transcription factors was further validated in SK-N-BE(2) cells by immunofluorescence assay. DAPI stain and phalloidin stain were used to label cell nucleus and cell cytoskeleton, respectively. Since tested stem cell marker proteins—sox2, oct4 and nanog are transcription factors, their signals should be in nucleus. Representative images (FIG. 3) demonstrated that the signals for sox2, oct4 and nanog were detected in nucleus, as these signals were overlapped with DAPI (not with phalloidin) in merged images. These results confirmed the presence of cancer stem cells in neuroblastoma cell lines.

### Metformin Lowers the Level of Stem Cell Marker Proteins

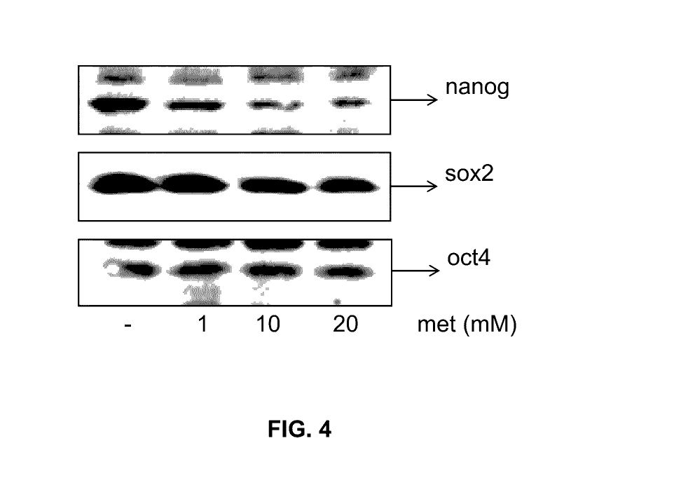

To confirm the effects of metform in on neuroblastoma stem cells the protein levels of sox2, oct4 and nanog in metform in-treated SK-N-BE(2) cells were examined. Two day metformin treated (1, 10 and 20 mM) SK-N-BE(2) cells were lysed, total cell proteins were prepared and separated on SDS-polyacrylamide gel. Western blots (FIG. 4) depicted that compared to metform in-untreated control group, the level of sox2 and nanog proteins was dose dependently decreased in metform in-treated samples (FIG. 4 upper two panels). No change in oct4 protein expression was observed among metformin-treated or -untreated samples (FIG. 4 lower panel). These results indicated that metformin reduces the levels of sox2 and nanog proteins in SK-N-BE(2) cells.

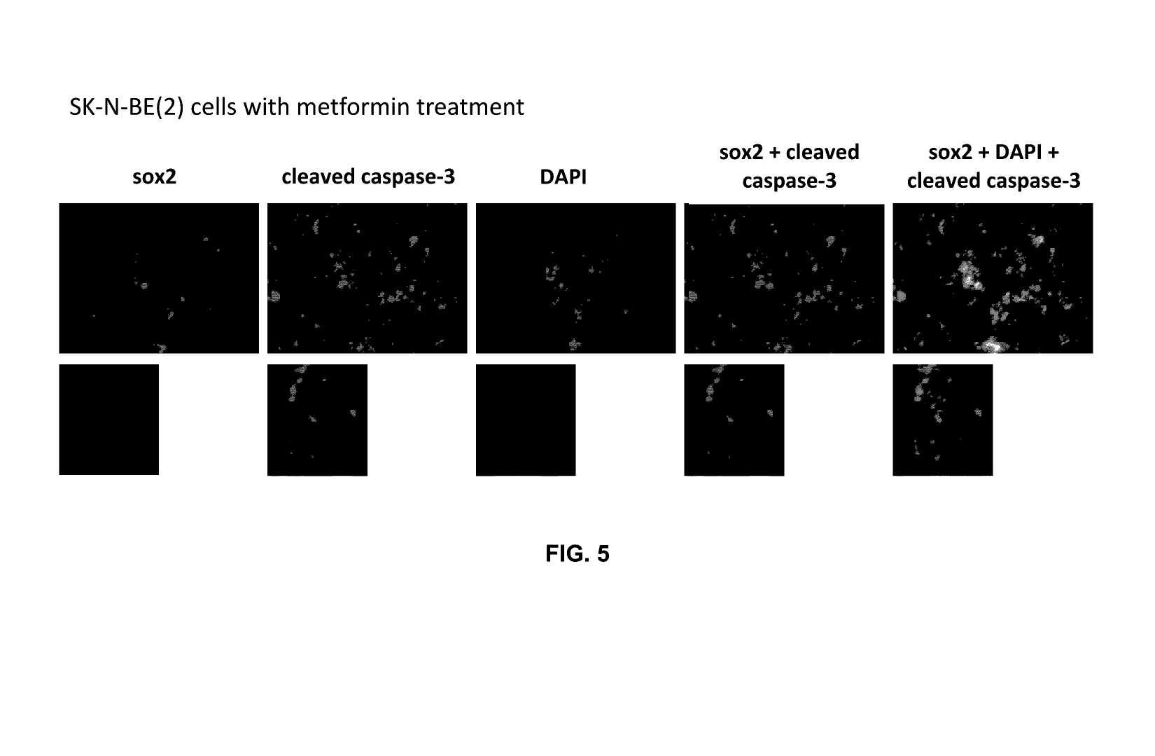

To confirm if metformin promotes apoptotic cell death in neuroblastoma stem cells, immunofluorescence staining was performed with antibodies against sox2 and active form of caspase-3 (cleaved caspase-3) using SK-N-BE(2) cells treated with metformin (10 mM) for 2 days. Caspase-3 is a precursor protein which after apoptotic signals cleaved into active form (termed cleaved caspase-3) that in turn relay signals to induce DNA fragmentations and ultimately cell death. Antibody used exclusively detects active form of caspase-3 (not cross-reacts with full length precursor caspase-3) in the cell. The immunofluorescence images (FIG. 5) demonstrated that cleaved caspase-3 signals are cytoplasmic, and present in both sox2-expressing and sox2-nonexpressing cells treated with metformin (See merged images in FIG. 5). These results confirm that metformin not only in normal cancer cells but also in neuroblastoma stem cells (sox2-expressing cells) induce apoptotic cell death.

### Metformin Reduces the Stemness of Stem Cells

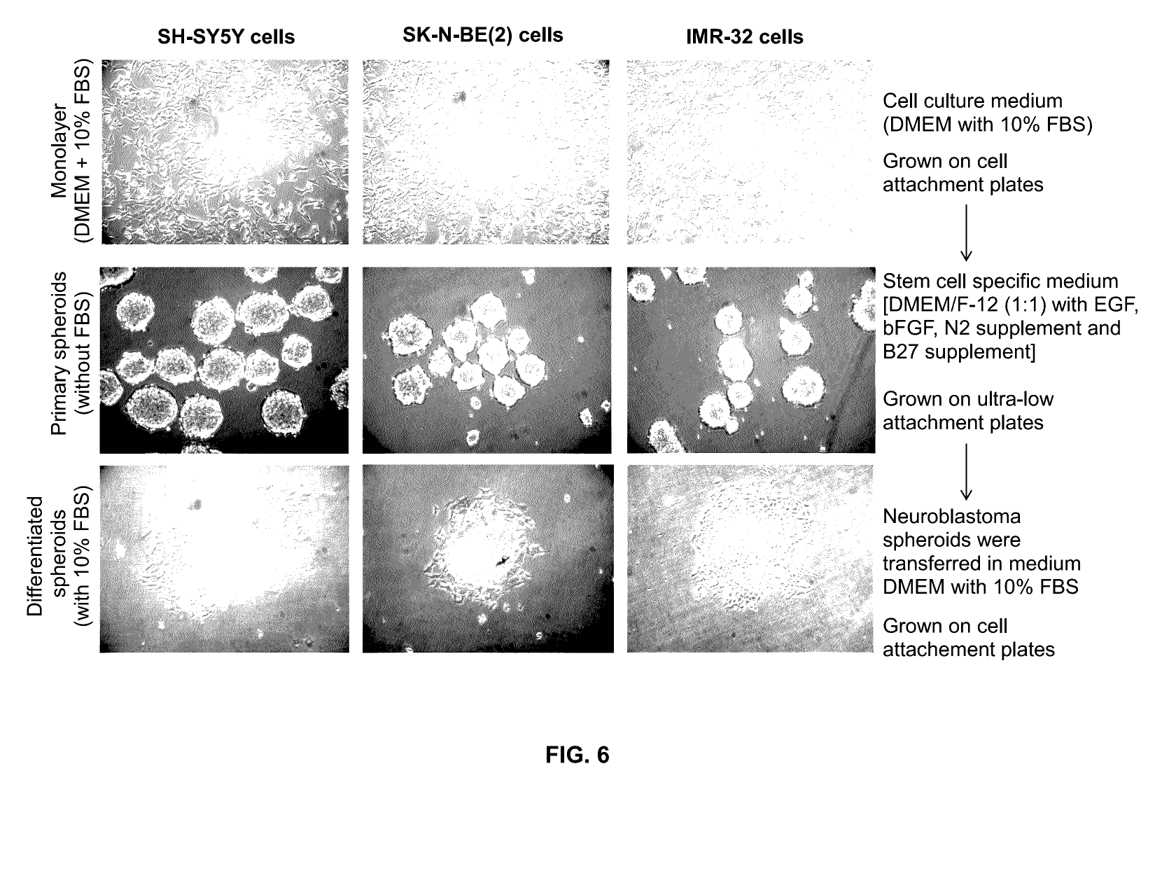

Stem cells form spheroids in stem cell specific medium supplemented with growth factors. Traditionally, neuroblastoma cell lines are maintained in medium containing 10% fetal bovine serum (FBS), and grown as monolayers in differentiated state (FIG. 6, upper panel). When cells are cultured in stem cell specific medium (DMEM/F-12 with EGF, bFGF, N-2 and B27 supplement) with no FBS, most of the tumor cells were floating and formed spheroids enriched with cancer stem cells (FIG. 6, middle panel). The formed spheroids were further differentiated and grown as monolayer if transferred in cell culture medium containing 10% FBS (FIG. 6, lower panel).

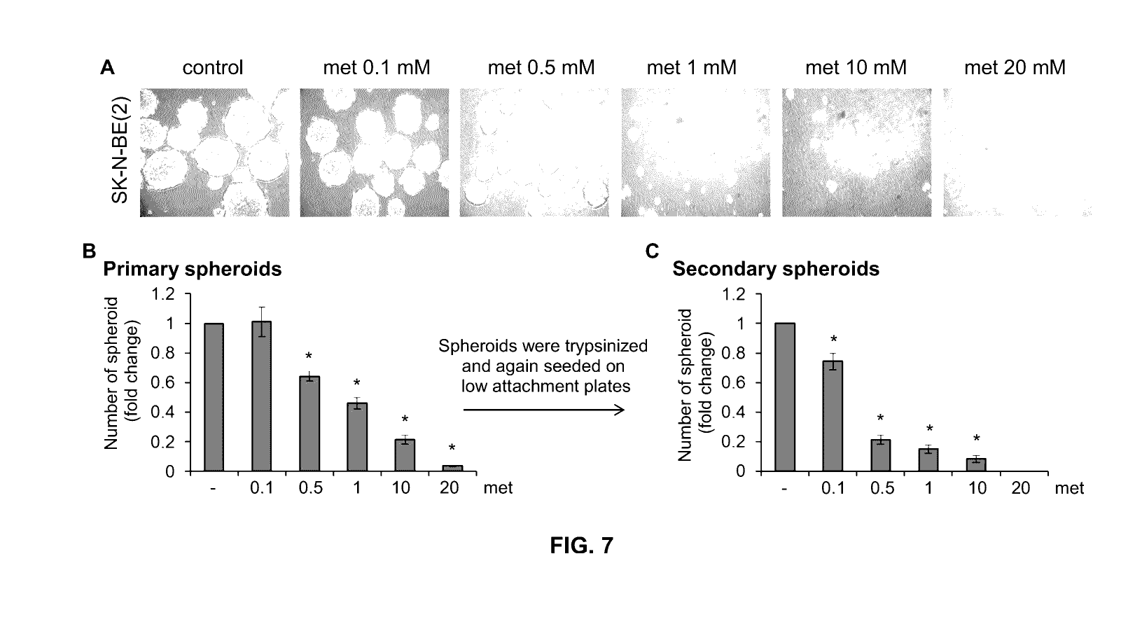

To test the effect of metformin in neuroblastoma spheroid formation, SK-N-BE(2) cells were grown in stem cell specific medium on ultra-low attachment plates and the growth of spheroid formation was monitored For spheroid formation assays, SK-N-BE(2) cells were seeded at density of 1000 cells per well in six-well plate in stem cell-specific medium with or without metformin (0.1, 0.5, 1, 10 and 20 mM). After 20 days, pictures of formed spheroids were taken and the number of spheroids equal to or larger than 100 μm in size (termed primary spheroids) was counted (FIG. 7 at A and B). After 20 days, distinct sphere formation was observed in the control, but metformin significantly decreased the formation of primary neuroblastoma spheroids in a dose dependent manner (FIG. 7 at A and B). The numbers of primary spheroids in the control and 0.1 mM metformin groups were similar while metformin at 0.5 mM and higher concentrations reduced primary spheroid formation significantly. To evaluate self-renewal or clonogenic potential of stem cells, primary spheroids from control and metformin-treatment groups were dissociated by trypsin and reseeded in 6-well ultra-low attachment plate (1,000 cells per well) in the presence of stem cell-specific medium with respective concentrations of metformin. Secondary spheroid formation (as an indicator of self-renewal potential of neuroblastoma spheres) was analyzed. After 20 days, the number of formed secondary spheroids (equal or larger than 100 μm in size) was counted and plotted (FIG. 7 at C). It was found that metformin even at lower concentration (0.1 mM) significantly inhibited secondary spheroid formation. These results confirm that metformin reduced both self-renewal and differentiation potential of neuroblastoma stem cells.

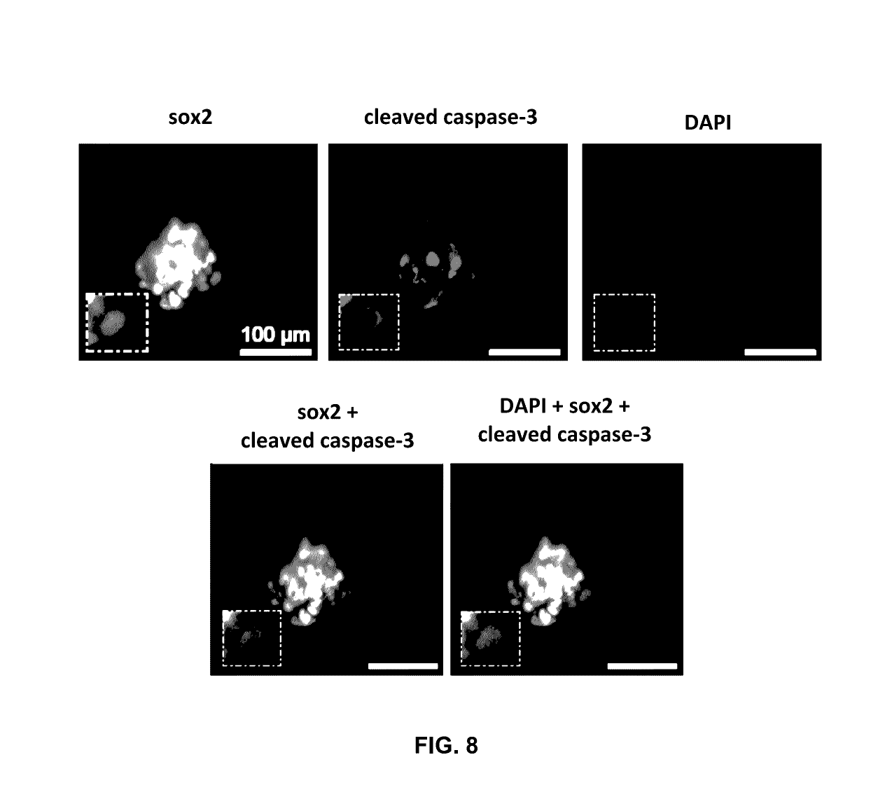

Since metformin significantly inhibited the growth of spheroids, the effect of metformin on spheroids at cellular level was further analyzed by detecting cleaved caspase-3 signals (immuno-fluorescence analysis). The presence of cleaved caspase-3 signal sox2-expressing cells confirmed that metformin induced apoptotic death in spheroids (FIG. 8).

Metformin Inhibits Growth of Tumors Generated from Neuroblastoma Spheroids

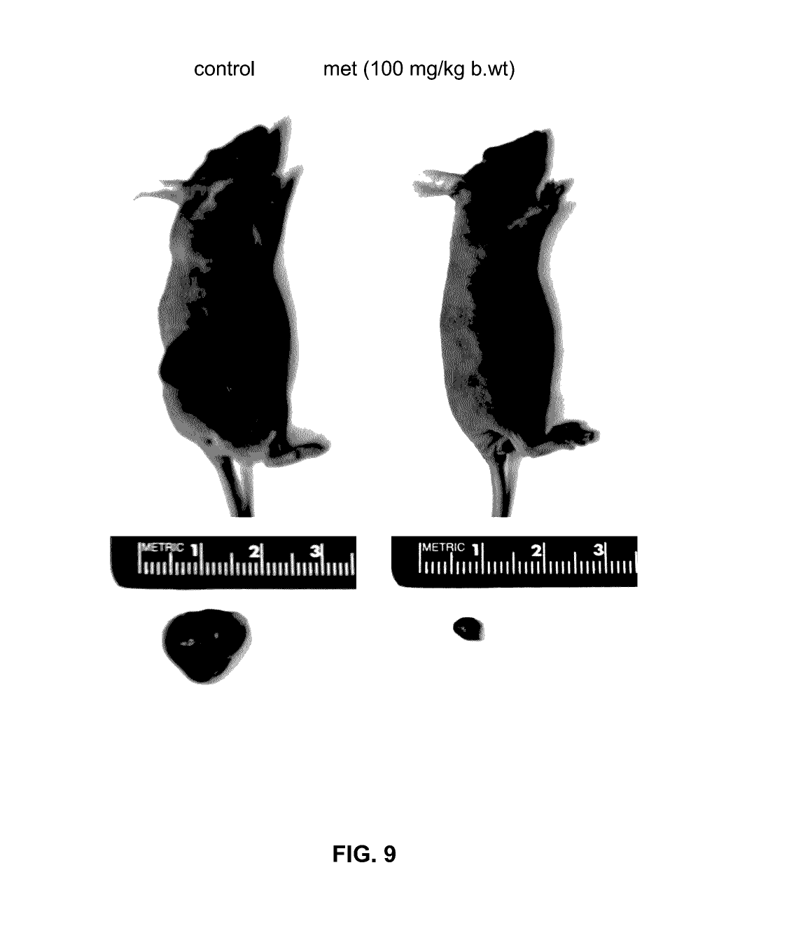

To assess whether metformin has inhibitory effects on the tumorigenicity of neuroblastoma stem cells, tumors were generated from neuroblastoma stem cells by inoculating tumor cells from SK-N-BE(2) spheroids (15-day-old) subcutaneously in to the flank region of athymic nude mice. Metformin (100 mg/kg per mice) was given daily by oral gavage when the palpable tumor was visible (˜10 days after inoculation). The size of tumors was measured on every fourth day, and after 21 days of metformin treatment tumors were harvested and sizes were measured. As demonstrated in FIG. 9, the size of the subcutaneous tumor was less in mice treated with metformin at dose of 100 mg/kg when compared with tumors from control metformin-untreated mice.

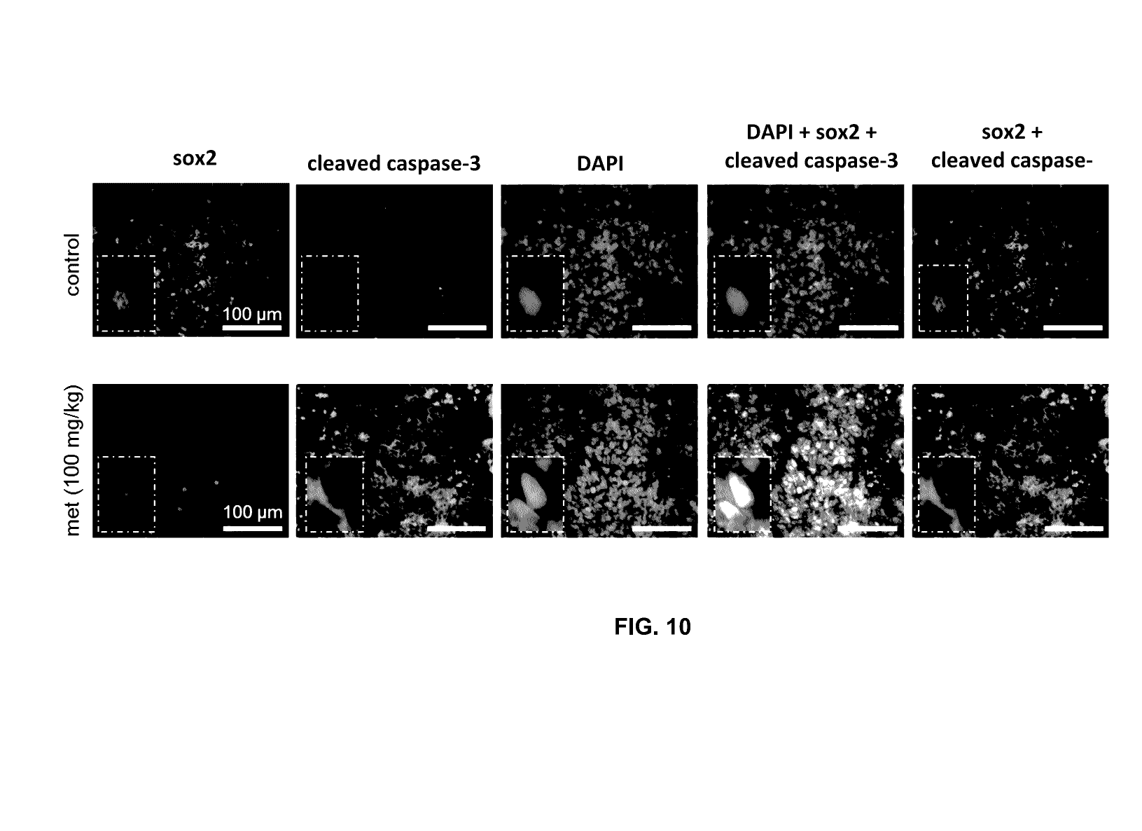

To determine if metformin inhibition of tumor growth resulted from apoptotic cell death in tumor stem cells, immunohistochemistry was performed by staining paraffin-embedded tumor sections with antibodies specific for active cleaved form of caspase-3 (an indicator of apoptosis) and stem cell specific marker sox2. The representative immunofluorescence images (FIG. 10) demonstrated that cytoplasmic cleaved caspase-3 signals were present in metformin-treated tumor tissues (FIG. 10, lower panel), but not in control metform in-untreated tumors (FIG. 10, upper panel). The presence of cleaved caspase-3 signal in both sox2-expressing and non-expressing cancer cells indicated that metformin induced cell death not only in normal tumor cells but also in tumor stem cells. These data further validated that metformin has anti-tumor activity against neuroblastoma stem cells.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they include structural elements that do not differ from the literal language of the claims, or if they include equivalent structural elements with insubstantial differences from the literal languages of the claims.

What is claimed is:

1. A method for inhibiting the growth and/or viability of neuroblastoma cells, the method comprising contacting neuroblastoma cells with metformin.

2. The method of claim 1, the neuroblastoma cells including one or more of SH-SY5Y cells, SK-N-BE(2) cells, IMR-32 cells, NGP cells and SK-N-F1 cells.

3. The method of claim 1, wherein the metformin is metformin hydrochloride.

4. The method of claim 1, wherein the metformin triggers apoptotic cell death pathways via activation of a caspase cascade.

5. A method for inhibiting the growth and/or viability of neuroblastoma stem cells, the method comprising contacting neuroblastoma stem cells with metformin.

6. The method of claim 5, the neuroblastoma stem cells including one or more of SH-SY5Y cells, SK-N-BE(2) cells, IMR-32 cells, NGP cells and SK-N-F1 cells.

7. The method of claim 5, wherein the metformin is metformin hydrochloride.

8. The method of claim 5, wherein the metformin triggers apoptotic cell death pathways via activation of a caspase cascade.

9. A method for treating neuroblastoma comprising delivering a composition to a subject in need thereof, the composition comprising metformin.

10. The method of claim 9, wherein the composition is delivered to the patient at a metformin dosage of from about 5 mg/kg/day to about 20 mg/kg/day.

11. The method of claim 9, wherein the composition is a solid.

12. The method of claim 9, wherein the composition is administered orally, topically, or as an implantable unit.

13. The method of claim 9, wherein the composition is a liquid.

14. The method of claim 9, wherein the composition is administered orally, topically, perenterally, or transdermally.

15. The method of claim 9, wherein the composition is delivered via a timed release or sustained release delivery system.

16. The method of claim 9, wherein the method is carried out in conjunction with one or more additional forms of therapy.

17. The method of claim 16, wherein the one or more additional forms of therapy comprise one or more of chemotherapy, radiotherapy, or surgical intervention.
